(19) United States
(12) Patent Application Publication
(10) Pub. No.: US 2021/0096117 A1
(43) Pub. Date: Apr. 1, 2021

(54) GAS SENSOR

(71) Applicant: NGK INSULATORS, LTD., Nagoya (JP)

(72) Inventors: Yusuke WATANABE, Nagoya (JP); Shotaro NIIZUMA, Kasugai (JP); Hayami AOTA, Nagoya (JP); Toshihiro HIRAKAWA, Kasugai (JP)

(21) Appl. No.: 17/030,506

(22) Filed: Sep. 24, 2020

(30) Foreign Application Priority Data
Sep. 27, 2019 (JP) 2019-177218

(51) Int. Cl. G01N 33/00

(52) U.S. Cl. CPC G01N 33/0036

(57) ABSTRACT

A gas sensor includes a sensor element; a plurality of contact members holding a rear end portion of the sensor element and being electrically connected to the sensor element; a plurality of lead wires that are fixed at a position rearward of the sensor element and to which the contact members are electrically connected; and metal elastic members fixing the contact members and the lead wires with the contact members and lead wires being spaced apart from each other, and electrically connecting the contact members and the lead wires.

### CROSS-REFERENCE TO RELATED APPLICATION

This application is based upon and claims the benefit of priority from Japanese Patent Application No. 2019-177218 filed on Sep. 27, 2019, the contents of which are incorporated herein by reference.

### BACKGROUND OF THE INVENTION

### Field of the Invention

The present invention relates to a gas sensor.

### Description of the Related Art

The technique disclosed in Japanese Laid-Open Patent Publication No. 2014-199721 has an object to provide a gas sensor that is less prone to short-circuiting of lead wires, metal terminal members, etc. and that provides stable sensor output.

In order to achieve the object above, the technique described in Japanese Laid-Open Patent Publication No. 2014-199721 restricts movements of lead wires (48) and contact fittings (71) by forming through holes (47a) inside a rubber stopper (47) and fixing the contact fittings (71) using a ceramic housing (51).

### SUMMARY OF THE INVENTION

In the gas sensor described in Japanese Laid-Open Patent Publication No. 2014-199721, the lead wires are directly joined to the contact members that are in direct contact with the sensor element to provide electric connection.

Accordingly, if the gas sensor vibrates beyond expectation, then force will act on the joined portions and may break the joined portions in the worst case. In particular, if the force concentrates in the area between the rear ends of the joined portions and the front ends of the coatings of the lead wires (the area where the lead wires are naked), then the gas sensor may vibrate around that area and may possibly break.

Further, when the sensor element is made shorter, heat from the sensor element may be transmitted to the lead wires and cause erosion (dissolved loss) of the coatings and elastic insulating material. Depending on the circumstances, factors like the vibrations mentioned above may cause contact failures.

The present invention has been made considering the problems above, and an object of the present invention is to provide a gas sensor capable of solving the problems and preventing breakage, contact failures, etc. from occurring even in long-term use.

A gas sensor according to an aspect of the present invention includes:

a sensor element;

a plurality of contact members configured to hold a rear end portion of the sensor element and be electrically connected to the sensor element;

a plurality of lead wires that are fixed at a position rearward of the sensor element and to which the contact members are electrically connected; and

at least one elastic member configured to fix the contact members and the lead wires with the contact members and the lead wires being spaced apart from each other, and electrically connect the contact members and the lead wires.

According to the present invention, it is possible to electrically connect the contact members and the lead wires in a state where they are spaced apart from each other, and also to elastically hold the contact members and the lead wires. This prevents breakage, contact failures, etc. from occurring even in long-term use.

The above and other objects, features, and advantages of the present invention will become more apparent from the following description when taken in conjunction with the accompanying drawings, in which a preferred embodiment of the present invention is shown by way of illustrative example.

### BRIEF DESCRIPTION OF THE DRAWINGS

FIG. 1 is a cross section illustrating a gas sensor according to an embodiment;

FIG. 2A is an explanatory diagram illustrating relative positioning of a tubular portion of an elastic member and a contact member, and relative positioning of a crimping portion of the elastic member and a lead wire; and FIG. 2B is a perspective view illustrating a state in which the contact member is wrapped by the tubular portion and the lead wire is crimped by the crimping portion.

### DESCRIPTION OF THE PREFERRED EMBODIMENTS

The gas sensor according to the present invention will be described below in detail in connection with preferred embodiments while referring to the accompanying drawings.

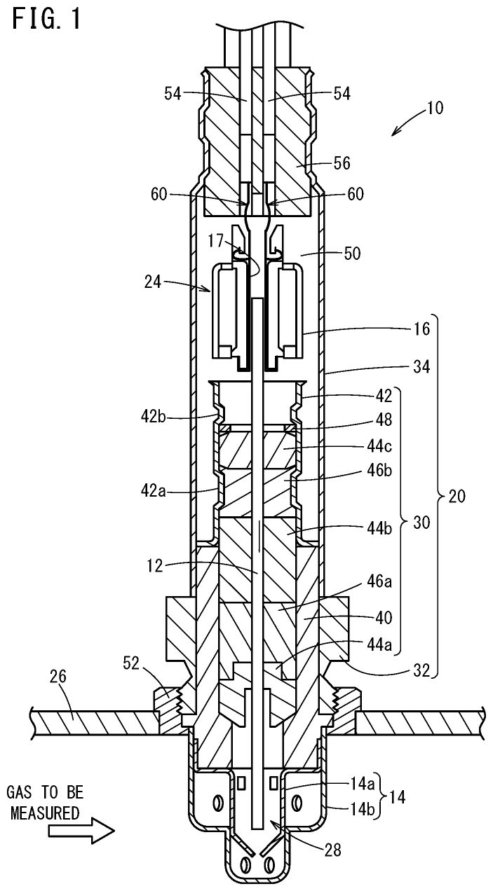

As shown in FIG. 1, a gas sensor 10 according to this embodiment includes a sensor element 12. The sensor element 12 has an elongated rectangular parallelepiped shape. The longitudinal direction of the sensor element 12 is defined as a front-rear direction, and the thickness direction of the sensor element 12 is defined as a top-bottom direction. The width direction of the sensor element 12 (a direction vertical to the front-rear and top-bottom directions) is defined as a left-right direction.

As shown in FIG. 1, the gas sensor 10 includes the sensor element 12, a protective cover 14 for protecting the front end of the sensor element 12, and a sensor assembly 20 including a ceramic housing 16. The ceramic housing 16 holds a rear end portion of the sensor element 12, and functions as a connector 24 by contact members 17 electrically connected to the sensor element 12 being attached thereto.

As shown in the drawing, the gas sensor 10 is attached to piping 26, such as an exhaust gas pipe of a vehicle, for example, and used to measure concentrations of specific gases, such as NOx, O₂, etc., that are contained in the exhaust gas, which is a gas to be measured (which will be also referred to as a measured gas).

The protective cover 14 includes a bottomed-tubular-shaped inner protective cover 14a covering the front end of the sensor element 12 and a bottomed-tubular-shaped outer protective cover 14b covering the inner protective cover 14a. The inner protective cover 14a and the outer protective cover 14b have formed therein a plurality of holes through which gas to be measured can flow into the interior of the protective cover 14. A sensor element chamber 28 is formed as a space enclosed by the inner protective cover 14a, and the front end of the sensor element 12 is disposed within the sensor element chamber 28.

The sensor assembly 20 includes an element seal body 30 for sealing and fixing the sensor element 12, a nut 32 attached to the element seal body 30, an outer tube 34, and the connector 24. The connector 24 is in contact with and electrically connected to electrodes (not shown) that are formed on the surfaces (top and bottom surfaces) of the rear end of the sensor element 12.

The element seal body 30 includes a tubular main fitting 40, and a tubular, inner tube 42 that is welded and fixed coaxially with the main fitting 40. The element seal body 30 includes ceramic supporters 44a to 44c, green compacts (pressurized powder body) 46a, 46b, and a metal ring 48 which are sealed in an inner through hole in the interior of the main fitting 40 and the inner tube 42. The sensor element 12 is located on the center axis of the element seal body 30 and penetrates through the element seal body 30 in the front-rear direction. The inner tube 42 has a reduced-diameter portion 42a and a reduced-diameter portion 42b. The reduced-diameter portion 42a presses the green compact 46b toward the center axis of the inner tube 42. The reduced-diameter portion 42b presses frontward the ceramic supporters 44a to 44c and the green compacts 46a, 46b through the metal ring 48. The pressing forces from the reduced-diameter portions 42a, 42b compress the green compacts 46a, 46b between the main fitting 40 and inner tube 42 and the sensor element 12. The green compacts 46a, 46b thus provide a seal between the sensor element chamber 28 in the protective cover 14 and a space 50 in the outer tube 34 and fix the sensor element 12.

The nut 32 is fixed coaxially with the main fitting 40, and has a male thread portion formed on its outer peripheral surface. The male thread portion of the nut 32 is inserted in a fixing member 52 that is welded to the piping 26 and has a female thread formed on its inner peripheral surface. The gas sensor 10 is thus fixed to the piping 26 with the front end of the sensor element 12 and the protective cover 14 projecting into the piping 26.

The outer tube 34 encloses the inner tube 42, the sensor element 12, and the connector 24. A plurality of lead wires 54 connected to the connector 24 are led out from the rear end of the outer tube. The lead wires 54 electrically conduct through the connector 24 to electrodes of the sensor element 12 (which will be described later). The gap between the outer tube 34 and the lead wires 54 is sealed by an elastic insulating member 56 formed from grommet or the like. The space 50 in the outer tube 34 is filled with a reference gas (the air in this embodiment). The rear end of the sensor element 12 is disposed within this space 50.

Now, in this embodiment, the contact members 17 extending rearward are electrically connected to corresponding connecting terminals (not shown) that are exposed at the rear end portion of the sensor element 12. In this embodiment, the ceramic housing 16 is provided around the rear end portion of the sensor element 12, and the contact members 17 are fitted in between the connecting terminals and the ceramic housing 16. The connecting terminals of the sensor element 12 and the contact members 17 are thus press fitted and electrically connected together. That is, the ceramic housing 16 is provided with the contact members 17 electrically connected to the sensor element 12 and holds the rear end portion of the sensor element 12.

The rear ends of the contact members 17 are electrically connected to corresponding lead wires 54 respectively through corresponding elastic members 60. The elastic members 60 are made of a metal material having electric conductivity and good elasticity, such as stainless steel, iron-Ni alloy, etc.

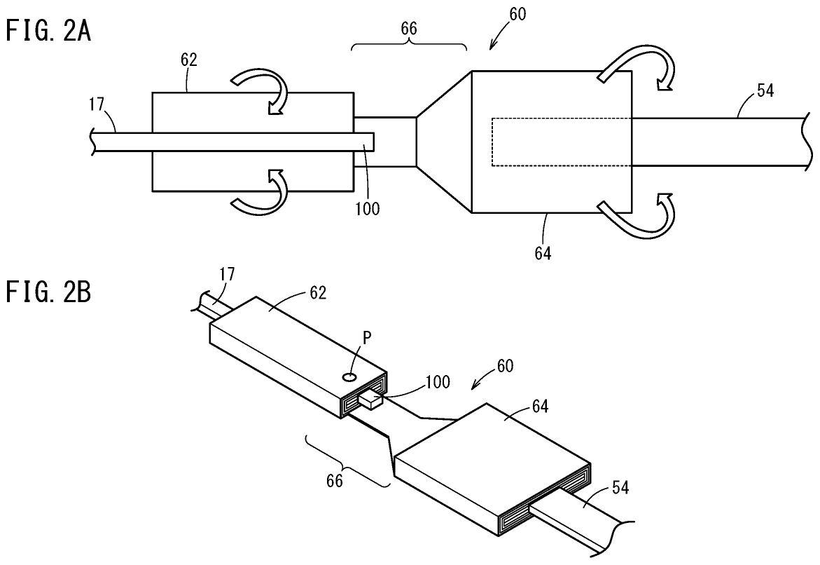

Now, referring to FIGS. 2A and 2B, an example will be representatively explained in which one contact member 17 is electrically connected to one lead wire 54 through one elastic member 60.

As shown in FIG. 2A, the elastic member 60 includes a tubular portion 62 through which the contact member 17 is inserted and which is electrically connected to the contact member 17, and a crimping portion 64 that crimps (or swages) the lead wire 54 and is electrically connected to the lead wire 54.

The elastic member 60 further includes an intermediate portion 66. The intermediate portion 66 is integrally formed between the tubular portion 62 and the crimping portion 64. The intermediate portion 66 extends from the tubular portion 62 to the crimping portion 64 and is formed so as to have a wide width.

Then, in the elastic member 60, the tubular portion 62 wraps the contact member 17 in one direction (i.e., toward the front of the drawing sheet of FIG. 2A). The tubular portion 62 wraps the contact member 17 with one end 100 of the contact member 17 being directed toward the lead wire 54 and with the end being positioned in the intermediate portion 66. In this case, the tubular portion 62 wraps the contact member 17 with the one end 100 of the contact member 17 protruding toward the lead wire 54.

Further, in the elastic member 60, the crimping portion 64 crimps the lead wire 54 in another direction (i.e., toward the back of the drawing sheet of FIG. 2A (in the depth direction)). The crimping portion 64 crimps the lead wire 54 with the end of the lead wire 54 not reaching the intermediate portion 66.

Further, as shown in FIG. 2B, the contact member 17 and the tubular portion 62 are welded together at a welded point P. That is, the welded point P is a joined portion when the elastic member 60 is formed into the tubular form. It is preferred that the welded point P be located on a rear end side of the tubular portion 62 (a side closer to the lead wire 54). The welding can be conducted by various methods, but spot welding is preferred.

### EXAMPLES

Now, one example experiment will be described. In the example experiment, a comparative example, example 1, and example 2 were subjected to vibration testing for 150 hours, with an acceleration of 50G and a frequency of 250 Hz, in order to check for breakage of contacts.

### Comparative Example

Comparative example has a conventional connection structure in which the contact member 17 and the lead wire 54 are directly connected by soldering.

### Example 1

Example 1 has a connection structure of the gas sensor 10 of the embodiment in which the contact member 17 and the lead wire 54 are connected by an elastic member, in particular a flat plate of metal. In the example 1, the contact member 17 and the flat plate are connected by spot welding and the flat plate and the lead wire 54 are connected by soldering.

### Example 2

Example 2 has a connection structure of the gas sensor 10 of the embodiment in which the contact member 17 and the lead wire 54 are connected by the elastic member 60. As shown in FIG. 2A, the elastic member 60 includes the tubular portion 62, the crimping portion 64, and the intermediate portion 66. In the example 2, the contact member 17 is wrapped by the tubular portion 62 in one direction and connected by spot welding, and the lead wire 54 is connected by being crimped in the crimping portion 64 in another direction.

### [Test Results]

The results of 150-hour vibration testing concerning the comparative example and examples 1 and 2 are shown below.

Comparative example: The sensor was examined after 150 hours have passed; breakage of contact members 17 and lead wires 54 was observed.

Example 1: No breakage occurred after 150 hours have passed, but deformation of the flat plates was observed.

Example 2: No breakage occurred after 150 hours have passed, and no deformation of the elastic members 60 was observed.

### Invention Obtained from Embodiments

The embodiments can be summarized as follows.

[1] A gas sensor 10 of the embodiment includes: a sensor element 12; a plurality of contact members 17 that holds a rear end portion of the sensor element 12 and is electrically connected to the sensor element 12; a plurality of lead wires 54 that are fixed at a position rearward of the sensor element 12 and to which the contact members 17 are electrically connected; and at least one metal elastic member 60 that fixes the contact members 17 and the lead wires 54 with the contact members 17 and the lead wires 54 being spaced apart from each other, and electrically connects the contact members 17 and the lead wires 54.

Thus, the elastic member 60 interposed between the contact member 17 and the lead wire 54 absorbs vibration and prevents breakage from occurring at the electric contact etc. Furthermore, interposing the elastic members 60 therebetween enlarges the distance between the sensor element 12 and the lead wires 54. This prevents erosion (dissolved loss) of components such as the elastic insulating member 56 through which the lead wires 54 are inserted.

[2] In the embodiment, the contact members 17 and the elastic members 60 are respectively joined together by welding. The elastic members 60 cancel out vibrations and thus prevent positional shifts of the contact members 17 and the lead wires 54.

[3] In the embodiment, the elastic members 60 each include: a tubular portion 62 through which the contact member 17 is inserted and that is electrically connected to the contact member 17; and a crimping portion 64 that crimps the lead wire 54 and is electrically connected to the lead wire 54.

Thus, the elastic member 60 interposed between the contact member 17 and the lead wire 54 absorbs vibration and thus prevents breakage from occurring at the electric contact etc. Furthermore, interposing the elastic members 60 therebetween enlarges the distance between the sensor element 12 and the lead wires 54. This prevents erosion (dissolved loss) of components such as the elastic insulating member 56 through which the lead wires 54 are inserted.

Furthermore, forming the elastic member 60 in a tubular shape increases the area of contact between the elastic member 60 and the contact member 17 and thus prevents contact failure. Similarly, providing the elastic member 60 with the crimping portion 64 electrically connected to the lead wire 54 increases the area of contact between the elastic member 60 and the lead wire 54 and thus prevents contact failure. As a result, contact failure between the contact member 17 and the lead wire 54 is prevented.

[4] In the embodiment, the contact members 17 are joined respectively to the tubular portions 62 by welding. This prevents positional shifts of the contact members 17 and the elastic members 60 caused by vibrations.

[5] In the embodiment, a joined portion of the joining by the welding is a joined portion of joining made when the elastic member 60 is formed into the tubular form. The portion joined by welding and the contact member 17 can be welded and fixed together, and thus the members can be joined together more firmly.

[6] In the embodiment, a joined portion of the joining by the welding is located on a rear end side of the tubular portion 62. Force concentrates in an area on the rear end side of the tubular portion 62, but providing the joined portion in this area provides reinforcement and enables the joined portion and the contact member 17 to be joined together more firmly.

[7] In the embodiment, the elastic members 60 each include an intermediate portion 66 integrally formed between the tubular portion 62 and the crimping portion 64. The intermediate portion 66 provides reinforcement between the tubular portion 62 and the crimping portion 64 to thereby prevent plastic deformation etc. of the elastic member 60.

[8] In the embodiment, the intermediate portion 66 extends from the tubular portion 62 to the crimping portion 64 and is formed so as to have a wide width. This reduces stress concentration from occurring during crimping of the lead wire 54, for example, and thus prevents plastic deformation etc. of the elastic member 60.

[9] In the embodiment, the crimping portion 64 crimps the lead wire 54 with an end of the lead wire 54 not reaching the intermediate portion 66. If the lead wire 54 is inserted up to the intermediate portion 66, the end portion of the lead wire 54 may cause deformation of the intermediate portion 66 at the time of crimping of the lead wire 54. As such, the lead wire 54 is placed so that its end does not reach the intermediate portion 66. As a result, the lead wire 54 can be crimped only in the crimping portion 64, which prevents the lead wire 54 from adversely affecting the intermediate portion 66.

[10] In the embodiment, the tubular portion 62 wraps the contact member 17 with one end of the contact member 17 protruding toward the lead wire 54. With this configuration, the end of the contact member 17 is exposed from the tubular portion 62, for example, when the contact member 17 and the tubular portion 62 are welded together. This enables the contact member 17 and the tubular portion 62 to be reliably welded together.

[11] In the embodiment, the direction in which the tubular portion 62 wraps the contact member 17 and the direction in which the crimping portion 64 crimps the lead wire 54 are different from each other. The tubular portion 62 and the crimping portion 64 provide an action similar to reverse crimping. This reduces warpage of the elastic member 60.

Implementations of the present invention may be provided with various means to improve reliability as an automotive component without departing from the idea of the present invention.

The present invention is not particularly limited to the embodiment described above, and various modifications are possible without departing from the essence and gist of the present invention.

What is claimed is:

1. A gas sensor comprising:
a sensor element;
a plurality of contact members configured to hold a rear end portion of the sensor element and be electrically connected to the sensor element;
a plurality of lead wires that are fixed at a position rearward of the sensor element and to which the contact members are electrically connected; and
at least one elastic member configured to fix the contact members and the lead wires with the contact members and the lead wires being spaced apart from each other, and electrically connect the contact members and the lead wires.

2. The gas sensor according to claim 1, wherein each of the contact members and the corresponding elastic member are joined together by welding.

3. The gas sensor according to claim 1, wherein the elastic member includes:
a tubular portion through which the contact member is inserted and that is electrically connected to the contact member; and
a crimping portion configured to crimp the lead wire and be electrically connected to the lead wire.

4. The gas sensor according to claim 3, wherein the contact members are joined respectively to the tubular portions by welding.

5. The gas sensor according to claim 4, wherein a joined portion of the joining by the welding is located on a rear end side of the tubular portion.

6. The gas sensor according to claim 4, wherein a joined portion of the joining by the welding is a joined portion of joining made when the elastic member is formed into a tubular form.

7. The gas sensor according to claim 6, wherein the joined portion of the joining by the welding is located on a rear end side of the tubular portion.

8. The gas sensor according to claim 3, wherein the elastic member includes an intermediate portion integrally formed between the tubular portion and the crimping portion.

9. The gas sensor according to claim 8, wherein the crimping portion crimps the lead wire with an end of the lead wire not reaching the intermediate portion.

10. The gas sensor according to claim 8, wherein the intermediate portion extends from the tubular portion to the crimping portion and is formed so as to have a wide width.

11. The gas sensor according to claim 10, wherein the crimping portion crimps the lead wire with an end of the lead wire not reaching the intermediate portion.

12. The gas sensor according to claim 3, wherein the tubular portion wraps the contact member with one end of the contact member protruding toward the lead wire.

13. The gas sensor according to claim 3, wherein a direction in which the tubular portion wraps the contact member and a direction in which the crimping portion crimps the lead wire are different from each other.
